(19) United States
(12) Patent Application Publication
(10) Pub. No.: US 2012/0122149 A1
(43) Pub. Date: May 17, 2012

(54) Absorbent bead concentration method that eliminates the need for centrifugation

(75) Inventor: Tanil Zuhtu Kocagoz, Istanbul (TR)

(73) Assignee: SALUBRIS, Inc

(21) Appl. No.: 13/199,776

(22) Filed: Sep. 9, 2011

(30) Foreign Application Priority Data
Sep. 15, 2010 (TR) 2010/07549

(51) Int. Cl. C12Q 1/04

(52) U.S. Cl. 435/34

(57) ABSTRACT

This invention is a technique that enables the concentration of cells, microorganisms, parasite eggs and cysts using absorbent beads, eliminating the need for centrifugation.

### CROSS REFERENCE TO RELATED APPLICATIONS

Turkish Application for Absorbent Bead Concentration Method that Eliminates the Need for Centrifugation:

Application No: 2010/07549

Filing Date: Sep. 15, 2010

Relationship of Applications: This is a Turkish application for the same invention to the Turkish Patent Institute.

### STATEMENT REGARDING FEDERALLY SPONSORED RESEARCH OR DEVELOPMENT

Not Applicable

### REFERENCE TO SEQUENCE LISTING

Not Applicable

### BACKGROUND OF INVENTION

The diagnosis of infectious diseases in humans and animals is made by identifying the causative organisms in samples like sputum, urine, feces, and cerebrospinal fluid by methods like microscopy, culture and molecular methods. In most of the classical methods, to increase the sensitivity, liquid or liquefied samples are centrifuged to sediment, thereby concentrating the infectious agents. Centrifugation requires extra time and effort. This invention enables the concentration of infectious agents by liquid absorbing beads, eliminating the need for centrifugation. Below, I will describe two versions of the method as examples of its use, one that enables concentration of parasites in stool samples and the other used for the diagnosis of tuberculosis that enables decontamination and concentration of clinical samples without centrifugation.

### BACKGROUND AND BRIEF SUMMARY OF THE INVENTION

This invention is a technique that enables the concentration of cells, microorganisms, parasite eggs and cysts using absorbent beads, eliminating the need for centrifugation.

Samples obtained from living organisms and from the environment are frequently examined for research purposes and for the diagnosis of diseases. Blood, sputum, stool, urine, cerebrospinal fluid, pleural fluid, peritoneal fluid, and stomach fluid obtained from both humans and animals are examples of frequently examined samples. If the number of cells, microorganisms, parasites, parasite eggs and cysts are low in the evaluated samples then it is difficult to identify these by diagnostic methods like microscopy, culture or molecular methods. To increase the sensitivity of detection, the most commonly used method is the concentration of cells, microorganisms, parasite eggs and cysts by sedimenting these using centrifugation of liquid or liquefied samples. After the organisms are sedimented by centrifugation the supernatant is discarded and the concentrated organisms that are in the sediment are suspended in a small volume of liquid. Examination of this concentrated liquid by microscopy increases the chance of revealing organisms, compared to direct examination of the original sample. Similarly, there is a greater chance of isolating organisms by culture or detection by molecular diagnostic methods using a concentrated sample.

Centrifugation is a time consuming procedure. Moreover, a centrifuge with appropriate features is required to spin biological samples. Using existing methods, sample concentration cannot be performed in laboratories where an appropriate centrifuge is not available. For concentration by centrifugation, first it is necessary to put the samples into appropriate centrifuge tubes. Then the tubes are placed in a counter-balanced way in the centrifuge rotor, and the tubes are then spun for several minutes. The supernatants are then discarded and finally the sediments must be resuspended. In addition to the time and effort expended, the extensive manipulation required by centrifugation poses a risk of infection to laboratory personnel with the infectious organisms that may be present in the samples.

This invention is a concentration method for biological samples that uses absorbent beads and thus eliminates the need for centrifugation. The absorbent beads used in this method may be, but are not limited to, agarose beads, silica gel, active alumina (aluminium oxide), aluminium silicate, active carbon, carbon molecular sieve and similar beads and crystals that have pores smaller than the organisms intended to be concentrated and that can absorb water and other liquids. When these beads are added to biological samples they quickly absorb the liquid part of the samples. Since the size of their pores is smaller than the organisms intended to be concentrated, the organisms cannot penetrate the beads during the absorption of the fluid. As most of the liquid is absorbed by the beads, the organisms become concentrated in the liquid that is left outside the beads.

The method of concentration using absorbent beads can be applied to any biological sample.

### BRIEF DESCRIPTION OF THE SEVERAL VIEWS OF THE DRAWING

Not Applicable

### DETAILED DESCRIPTION OF THE INVENTION

As noted above, the method of concentration using absorbent beads can be applied to any biological sample. Two examples are described in detail below, one for concentrating parasite trophozoites, cyst and eggs in stool samples and another for concentrating mycobacteria in sputum and other samples. The method is not restricted to these applications.

The new method is a vast improvement over the current method of sample concentration. Stool samples are commonly examined biological specimens. They are examined by microscopy for the diagnosis of intestinal parasites. In classical methods, the stool is first diluted and homogenized with a fixative solution like formaldehyde. (Formaldehyde helps to preserve the parasitic formations in their original shape.) Large, non-homogenized particles are then filtered using gauze or plastic filter. The filtrate is placed in centrifuge tubes and spun to sediment the parasite eggs, cysts and trophozoites (the active living form of the parasites). The supernatant is then discarded. The sediment is suspended in a small volume of fluid. The organisms are usually stained with iodine solution and then examined under the microscope for the presence of parasite cysts and eggs. It is hard to observe the trophozoites using this method since most of them are destroyed during centrifugation.

Using the absorbent bead method, the stool sample is diluted and homogenized by a fixative solution like formaldehyde-sodium acetate or formaldeyde-ethyl acetate or formaldehyde-detergent mixture. Staining solutions can also be added at this stage to stain parasitic organisms during the process. Then, absorbent beads are added into the homogenized sample and kept for a few minutes to allow for the absorption of most of the liquid. The concentrated sample is stained (if not stained during the process) and transferred to a slide and examined under the microscope for the presence of parasitic organisms. The deletion of the need for centrifugation in this case not only simplifies the process and reduces the danger of lab worker exposure to pathogens, it avoids destruction of the trophozoites, which concentration seeks to maximize in the sample.

Another common area to which the method can be applied is testing for Tuberculosis. Tuberculosis is an infectious disease of humans caused by a microorganism named Mycobacterium tuberculosis (tuberculosis bacilli). The most common active infection involves the lungs. The diagnosis of pulmonary tuberculosis is established by identifying Mycobacterium tuberculosis in sputum samples of the patients by microscopy and culture. Mycobacterium tuberculosis is a fairly slow growing organism in culture media. If sputum samples are directly inoculated to culture media, rapid growing organisms in the sample will grow and cover the surface of the media inhibiting the growth and observation of Mycobacterium tuberculosis. To eliminate this problem, sputum and any samples suspected to contain other organisms are decontaminated before being inoculated to culture media. Decontamination is a procedure that kills other organisms without killing Mycobacterium tuberculosis. For decontamination, first a decontamination solution is added to the sample, incubated for several minutes after which a neutralizing solution is added. Since the addition of these solutions dilutes the samples extensively, it is necessary to concentrate the tuberculosis bacilli, if they are present in the samples. The classical method requires centrifugation to accomplish sedimentation and thus concentration. For centrifugation, the decontaminated and neutralized samples must be placed in centrifuge tubes and spun in refrigerated centrifuges, (since the heat produced during centrifugation may kill tuberculosis bacilli).

In the system that is based on this invention, samples like sputum are homogenized and incubated in a cup with a decontamination solution. During incubation other organisms are killed while tuberculosis bacilli survive. Then absorbent beads are added into the sample. While the beads completely absorb the decontamination solution, tuberculosis bacilli stay on the surface of the beads since the pores of the beads are smaller than these bacilli. Then, a neutralizing solution which stops the killing effect of the decontamination solution is added to the sample. A portion of this solution is also absorbed by the beads. If present in the original sample, tuberculosis bacilli are concentrated in a small volume of fluid left around the beads. Since most of the solutions used in the procedure are absorbed by absorbent beads, the need for a refrigerated centrifuge, centrifugation and discarding the supernatant is eliminated. The absorbent bead method saves time and effort and reduces the danger of contact with and contamination of laboratory personnel with infectious organisms.

The above are merely examples of the use of the absorbent bead method. Concentration by absorbent beads can be applied to any biological samples when excess fluid is required to be eliminated and cells and disease causing organisms need to be concentrated.

1. Method for concentration of organisms in biological liquid or liquefied samples by absorbing the fluid with absorbent beads and thus concentrating and enabling the easy identification of disease causing cells and organisms without the need for centrifugation.

2. The absorbent beads in claim 1 may be, but are not limited to, spherical or other shaped crystals or beads like agarose beads, silica gel, active alumina (aluminium oxide), aluminum silicate, active carbon or carbon molecular sieve which have the characteristic of absorbing water and other fluids with pores smaller than organisms like bacteria, fungi, protozoa or helminth eggs.
